(19) United States
(12) Patent Application Publication
(10) Pub. No.: US 2026/0174659 A1
(43) Pub. Date: Jun. 25, 2026

(54) COMPOSITION COMPRISING LARGE AMOUNT OF INORGANIC UV FILTER AND ORGANIC UV FILTER SELECTED FROM ORGANOSILICON COMPOUNDS WITH BENZOTRIAZOLE GROUP

(71) Applicant: L'OREAL, Paris (FR)

(72) Inventor: Toru KOIKE, Kawasaki-shi (JP)

(21) Appl. No.: 19/126,301

(22) PCT Filed: Nov. 14, 2023
(86) PCT No.: PCT/JP2023/041556

§ 371 Date: May 1, 2025

(30) Foreign Application Priority Data
Nov. 30, 2022 (JP) 2022-191792
Jan. 6, 2023 (FR) 2300150

(51) Int. Cl. A61Q 17/04; A61Q 19/00

(52) U.S. Cl. CPC A61K 8/585; A61K 8/27; A61K 8/29; A61Q 17/04

(57) ABSTRACT

A composition contains: (a) at least one inorganic UV filter, and (b) at least one organic UV filter selected from organosilicon compounds having at least one benzotriazole group. The amount of the (a) inorganic UV filter is more than 5% by weight relative to the total weight of the composition. A composition can provide sufficient UV shielding effects as well as a smooth feeling to touch, even though it includes a relatively large amount of inorganic UV filter.

### TECHNICAL FIELD

The present invention relates to a composition including a relatively large amount of inorganic UV filter and at least one organic UV filter selected from organosilicon compounds with at least one benzotriazole group, as well as a cosmetic process using the composition and a use of the organic UV filter in a composition comprising a relatively large amount of inorganic UV filter.

### BACKGROUND ART

Many cosmetics include one or more UV filters in order to shield UV rays. In particular, skin cosmetics commonly include organic and/or inorganic UV filters for protecting the skin from UV rays. Since various types of damage to the skin such as wrinkles caused by UV rays are now well recognized, the need for UV care cosmetics has further increased.

### DISCLOSURE OF INVENTION

However, it has been discovered that if a composition includes a relatively large amount of inorganic UV filter such as TiO₂, the composition tends to provide a squeaky texture.

Thus, an objective of the present invention is to provide a composition which can provide sufficient UV shielding effects as well as a smooth feeling to touch, even though it includes a relatively large amount of inorganic UV filter.

The above objective of the present invention can be achieved by a composition comprising:
(a) at least one inorganic UV filter, and
(b) at least one organic UV filter selected from organosilicon compounds having at least one benzotriazole group,
   wherein
   the amount of the (a) inorganic UV filter is more than 5% by weight relative to the total weight of the composition.

The (a) inorganic UV filter may be selected from titanium dioxide, zinc oxide and a mixture thereof.

The amount of the (a) inorganic UV filter(s) in the composition according to the present invention may be 20% by weight or less, preferably 15% by weight or less, and more preferably 10% by weight or less, relative to the total weight of the composition.

The (b) organic UV filter may be selected from silanes and polyorganosiloxanes containing at least one structural unit having the formula (1):

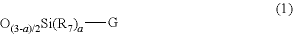

in which
R⁷ is an optionally halogenated C₁-C₁₀ alkyl radical, a phenyl radical, or a trimethylsilyloxy radical;
a is an integer ranging from 0 to 3, inclusive; and
G is a monovalent radical directly bonded to a silicon atom and having the structural formula (2):

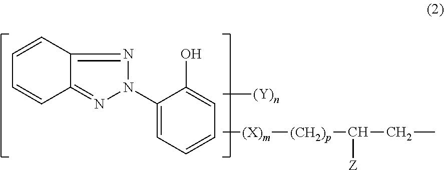

in which
the radicals Y, which may be identical or different, are each a C₁-C₈ alkyl radical, a halogen atom, or a C₁-C₄ alkoxy radical, with the proviso that, in the latter instance, two adjacent radicals Y on the same aromatic ring can together form an alkylidenedioxy radical wherein the alkylidene moiety has 1 or 2 carbon atoms;
X is O or NH;
Z is hydrogen or a C₁-C₄ alkyl radical;
n is an integer ranging from 0 to 3, inclusive;
m is O or 1; and
p is an integer ranging from 1 to 10, inclusive.

The (b) organic UV filter may be selected from benzotriazole-substituted silicone compounds having either of the structural formulae (5) and (6):

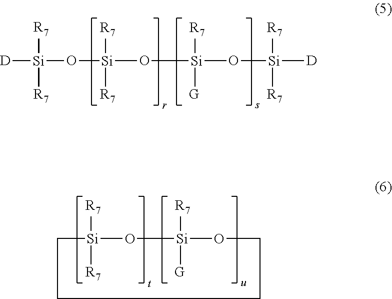

in which
the radicals R⁷, which may be identical or different, are each a C₁-C₁₁ alkyl, phenyl, 3,3,3-trifluoropropyl, or trimethylsilyloxy radical, at least 80% by number of the radicals R⁷ being methyl radicals;
the radicals D, which may be identical or different, are each a radical R⁷ or a radical G; r is an integer ranging from 0 to 50, inclusive, and s is an integer ranging from 0 to 20, inclusive, with the proviso that if s=0, at least one of the two radicals D is a radical G; u is an integer ranging from 1 to 6, inclusive, and t is an integer ranging from 0 to 10, inclusive, with the proviso that t+u is equal to or greater than 3; and the radical G has the above structural formula (2).

The (b) organic UV filter may be selected from benzotriazole-substituted silicone compounds having the structural formula (7):

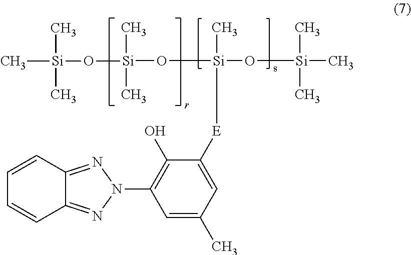

in which 0≤r≤10; 1≤s≤10; and E is the divalent radical:

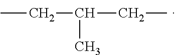

The (b) organic UV filter may be drometrizole trisiloxane having the structural formula:

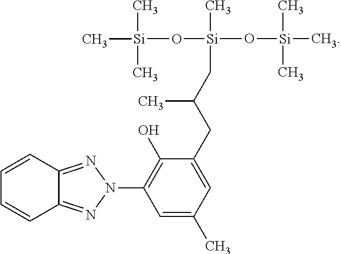 [SMILES: Cc1cc(CC(C)C[Si](C)(O[Si](C)(C)C)O[Si](C)(C)C)c(O)c(-n2nc3ccccc3n2)c1]

The amount of the (b) organic UV filter(s) in the composition according to the present invention may be from 1% to 20% by weight, preferably from 2% to 15% by weight, and more preferably from 3% to 10% by weight, relative to the total weight of the composition.

The composition according to the present invention may further comprise (c) at least one fatty material.

The amount of the (c) fatty material(s) in the composition according to the present invention may be from 1% to 20% by weight, preferably from 3% to 15% by weight, and more preferably from 5% to 10% by weight, relative to the total weight of the composition.

The composition according to the present invention may further comprise (d) at least one phosphate compound.

The amount of the (d) phosphate compound(s) in the composition according to the present invention may be from 0.01% to 15% by weight, preferably from 0.05% to 10% by weight, and more preferably from 0.1% to 5% by weight, relative to the total weight of the composition.

The composition according to the present invention may be a cosmetic composition, preferably a skin cosmetic composition, and more preferably a skin care composition or a skin makeup composition.

The present invention also relates to a cosmetic process for a keratin substrate such as skin, comprising applying, to the keratin substrate, the composition according to the present invention.

The present invention also relates to a use of the (b) at least one organic UV filter selected from organosilicon compounds having at least one benzotriazole group in composition comprising the (a) at least one inorganic UV filter in an amount of more than 5% by weight relative to the total weight of the composition, in order to improve the smooth texture of the composition.

### BEST MODE FOR CARRYING OUT THE INVENTION

After diligent research, the inventors have discovered that it is possible to provide a composition which can provide sufficient UV shielding effects as well as a smooth feeling to touch, even though it includes a relatively large amount of inorganic UV filter.

Thus, the composition according to the present invention comprises:
(a) at least one inorganic UV filter, and
(b) at least one organic UV filter selected from organosilicon compounds having at least one benzotriazole group,
   wherein

the amount of the (a) inorganic UV filter is more than 5% by weight relative to the total weight of the composition.

The composition according to the present invention can provide sufficient UV shielding effects as well as a smooth feeling to touch, even though it includes a relatively large amount of inorganic UV filter.

The composition according to the present invention can provide sufficient UV shielding effects, in particular, not only UVA shielding effects but also UVB shielding effects.

The composition according to the present invention can reduce a squeaky feeling due to the relatively large amount of the (a) inorganic UV filter in the composition.

The composition according to the present invention can provide a smooth feeling to touch during the application of the composition onto a keratin substance such as skin.

The composition according to the present invention can also provide a smooth feeling to touch even after rinsing off the composition from a keratin substance such as skin.

The composition according to the present invention is also stable such that it is homogeneous or no crystal of the (b) organic UV filter is present.

Hereinafter, the composition, process, use and the like according to the present invention will be explained in a more detailed manner.

### [Inorganic UV Filter]

The composition according to the present invention includes (a) at least one inorganic UV filter. If two or more inorganic UV filters are used, they may be the same or different, preferably the same.

The (a) inorganic UV filter used for the present invention may be active in the UV-A and/or UV-B region. The (a) inorganic UV filter used for the present invention is water-insoluble in solvents such as water and ethanol commonly used in cosmetics, but may be hydrophilic and/or lipophilic.

It is preferable that the (a) inorganic UV filter be in the form of a fine particle such that the mean (primary) particle diameter thereof ranges from 1 nm to 50 nm, preferably 5 nm to 40 nm, and more preferably 10 nm to 30 nm. The mean (primary) particle size or mean (primary) particle diameter here is an arithmetic mean diameter.

The (a) inorganic UV filter can be selected from the group consisting of silicon carbide, metal oxides which may or may not be coated, and mixtures thereof.

Preferably, the (a) inorganic UV filters may be selected from pigments (mean size of the 30 primary particles: generally from 5 nm to 50 nm, preferably from 10 nm to 50 nm) formed of metal oxides, such as, for example, pigments formed of titanium oxide (amorphous or crystalline in the rutile and/or anatase form), iron oxide, zinc oxide, zirconium oxide or cerium oxide, which are all UV photoprotective agents that are well known per se.

Preferably, the (a) inorganic UV filters may be selected from titanium dioxide (TiO₂), zinc oxide, and more preferably titanium oxide.

The (a) inorganic UV filter may or may not be coated. The (a) inorganic UV filter may have at least one coating. The coating may comprise at least one compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes such as beeswax, (meth)acrylic polymers, organic UV filters, and (per)fluoro compounds.

It is preferable for the coating to include at least one organic UV filter. As the organic UV filter in the coating, a dibenzoylmethane derivative such as butyl methoxydibenzoylmethane (Avobenzone) and 2,2′-Methylenebis[6-(2H-Benzotriazol-2-yl)-4-(1,1,3,3-Tetramethyl-Butyl)Phenol](Methylene Bis-Benzotriazolyl Tetramethylbutylphenol) marketed as “TINOSORB M” by BASF may be preferable.

In a known manner, the silicones in the coating(s) may be organosilicon polymers or oligomers comprising a linear or cyclic and branched or cross-linked structure, of variable molecular weight, obtained by polymerization and/or polycondensation of suitable functional silanes and essentially composed of repeated main units in which the silicon atoms are connected to one another via oxygen atoms (siloxane bond), optionally substituted hydrocarbon radicals being connected directly to said silicon atoms via a carbon atom.

The term “silicones” also encompasses silanes necessary for their preparation, in particular alkylsilanes.

The silicones used for the coating(s) can preferably be selected from the group consisting of alkylsilanes, polydialkylsiloxanes, and polyalkylhydrosiloxanes. More preferably still, the silicones are selected from the group consisting of octyltrimethylsilanes, polydimethylsiloxanes, and polymethylhydrosiloxanes.

Of course, the inorganic UV filters made of metal oxides may, before their treatment with silicones, have been treated with other surfacing agents, in particular, with cerium oxide, alumina, silica, aluminum compounds, silicon compounds, or mixtures thereof.

The coated inorganic UV filter may have been prepared by subjecting the inorganic UV filter to one or more surface treatments of a chemical, electronic, mechanochemical, and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic UV filters may be titanium oxides coated with:
silica, such as the product “Sunveil” from Ikeda;
silica and iron oxide, such as the product “Sunveil F” from Ikeda;
silica and alumina, such as the products “Microtitanium Dioxide MT 500 SA” from Tayca, “Tioveil” from Tioxide, and “Mirasun TiW 60” from Rhodia;
alumina, such as the products “Tipaque TTO-55 (B)” and “Tipaque TTO-55 (A)” from Ishihara, and “UVT 14/4” from Kemira;
alumina and aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck;
alumina and aluminum laurate, such as the product “Microtitanium Dioxide MT 100 S” from Tayca;
iron oxide and iron stearate, such as the product “Microtitanium Dioxide MT 100 F” from Tayca;
zinc oxide and zinc stearate, such as the product “BR351” from Tayca;
silica and alumina and treated with a silicone, such as the products “Microtitanium Dioxide MT 600 SAS”, “Microtitanium Dioxide MT 500 SAS”, and “Microtitanium Dioxide MT 100 SAS” from Tayca;
silica, alumina, and aluminum stearate and treated with a silicone, such as the product “STT-30-DS” from Titan Kogyo;
silica and treated with a silicone, such as the product “UV-Titan X 195” from Kemira; alumina and treated with a silicone, such as the products “Tipaque TTO-55 (S)” from Ishihara or “UV Titan M 262” from Kemira;
triethanolamine, such as the product “STT-65-S” from Titan Kogyo;
stearic acid, such as the product “Tipaque TTO-55 (C)” from Ishihara; or
sodium hexametaphosphate, such as the product “Microtitanium Dioxide MT 150 W” from Tayca.

Other titanium oxide pigments treated with a silicone are preferably TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO₂Si₃” by Cardre, and anatase/rutile TiO₂ treated with a polydimethylhydrosiloxane and for which the mean size of the individual particles is 25 nm, such as that marketed under the trademark “Microtitanium Dioxide USP Grade Hydrophobic” by Color Techniques.

Preferably, the following coated TiO₂ can be used as the coated inorganic UV filter: Stearic acid (and) Aluminum Hydroxide (and) TiO₂, such as the product “MT-100 TV” from Tayca, with a mean primary particle diameter of 15 nm;

Dimethicone (and) Stearic Acid (and) Aluminum Hydroxide (and) TiO₂, such as the product “SA-TTO-S4” from Miyoshi Kasei, with a mean primary particle diameter of 15 nm; Silica (and) TiO₂, such as the product “MT-100 WP” from Tayca, with a mean primary particle diameter of 15 nm;

Dimethicone (and) Silica (and) Aluminum Hydroxide (and) TiO₂, such as the product “MT-Y02” and “MT-Y-110 M3S” from Tayca, with a mean primary particle diameter of 10 nm; Dimethicone (and) Aluminum Hydroxide (and) TiO₂, such as the product “SA-TTO-S3” from Miyoshi Kasei, with a mean primary particle diameter of 15 nm; Dimethicone (and) Alumina (and) TiO₂, such as the product “UV TITAN M170” from Sachtleben, with a mean primary particle diameter of 15 nm; and Silica (and) Aluminum Hydroxide (and) Alginic Acid (and) TiO₂, such as the product “MT-100 AQ” from Tayca, with a mean primary particle diameter of 15 nm.

In terms of UV filtering ability, TiO₂ coated with at least one organic UV filter is more preferable. For example, Avobenzone (and) Stearic Acid (and) Aluminum Hydroxide (and) TiO₂, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS”, and by Tioxide under the trademark “Tioveil AQ”.

The uncoated zinc oxide pigments are, for example:
those marketed under the trademark “Z-cote” by Sunsmart;
those marketed under the trademark “Nanox” by Elementis; and
those marketed under the trademark “Nanogard WCD 2025” by Nanophase Technologies.

The coated zinc oxide pigments are, for example:
those marketed under the trademark “Oxide Zinc CS-5” by Toshiba (ZnO coated with polymethylhydrosiloxane);
those marketed under the trademark “Nanogard Zinc Oxide FN” by Nanophase Technologies (as a 40% dispersion in Finsolv TN, C₁₂-C₁₅ alkyl benzoate);
those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); those marketed under the trademark “NFD Ultrafine ZnO” by Daikin (ZnO coated with
phosphate of perfluoroalkyl and a copolymer based on perfluoroalkylethyl as a dispersion in cyclopentasiloxane);
those marketed under the trademark “SPD-Z1” by Shin-Etsu (ZnO coated with a silicone-grafted acrylic polymer dispersed in cyclodimethylsiloxane);
those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C₁₂-C₁₅ alkyl benzoate with hydroxystearic acid polycondensate).

The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

Mention may also be made of mixtures of metal oxides, in particular, of titanium dioxide and of cerium dioxide, including a mixture of equal weights of titanium dioxide coated with silica and of cerium dioxide coated with silica marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica, and with glycerol, such as the product “M 211” marketed by Kemira.

Coated inorganic UV filters are preferable, because the UV filtering effects of the inorganic UV filters can be enhanced. In addition, the coating(s) may help uniformly or homogeneously disperse the UV filters in the composition according to the present invention.

The amount of the (a) inorganic UV filter(s) in the composition according to the present invention is more than 5% by weight, preferably 6% by weight or more, and more preferably 7% by weight or more, relative to the total weight of the composition.

The amount of the (a) inorganic UV filter(s) in the composition according to the present invention may be 20% by weight or less, preferably 15% by weight or less, and more preferably 10% by weight or less, relative to the total weight of the composition.

The amount of the (a) inorganic UV filter(s) in the composition according to the present invention may be more than 5% by weight and 20% by weight or less, preferably from 6% to 15% by weight, and more preferably from 7% to 10% by weight, relative to the total weight of the composition.

### [Organic UV Filter]

The composition according to the present invention includes (b) at least one organic UV filter selected from organosilicon compounds having at least one benzotriazole group. If two or more organic UV filters are used, they may be the same or different, preferably the same.

The (b) organic UV filter is selected from organosilicon compounds having at least one benzotriazole group.

The organosilicon compounds having at least one benzotriazole group may be silanes or siloxanes with a benzotriazole functional group comprising at least one structural unit of the following formula (1):

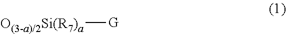

wherein
R⁷ denotes an optionally halogenated, C₁-C₁₀ alkyl radical, a phenyl radical, or a trimethylsilyloxy radical;
a denotes an integer ranging from 0 to 3, inclusive; and
G denotes a monovalent radical directly bonded to a silicon atom and having the structural formula (2) below:

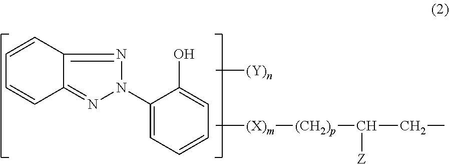

wherein
the radicals Y, which may be identical or different, are each a C₁-C₈ alkyl radical, a halogen atom, or a C₁-C₄ alkoxy radical, with the proviso that, in the latter instance, two adjacent radicals Y on the same aromatic ring can together form an alkylidenedioxy radical wherein the alkylidene moiety has 1 or 2 carbon atoms;
X is 0 or NH;
Z is hydrogen or a C₁-C₄ alkyl radical;
n is an integer ranging from 0 to 3, inclusive;
m is 0 or 1; and
p is an integer ranging from 1 to 10, inclusive.

These compounds are described in particular, in EP-A-0,392,882; EP-A-0,660,701; EP-A-0,708,108; EP-A-0,711,778; and EP-A-711,779.

The organosilicon compounds having at least one benzotriazole group may preferably belong to the general family of benzotriazole silicones which is described, in particular, in EP-A-0,660,701.

One family of benzotriazole silicones which is particularly preferred according to the present invention is that which includes the compounds corresponding to structural formula (5) or (6) below:

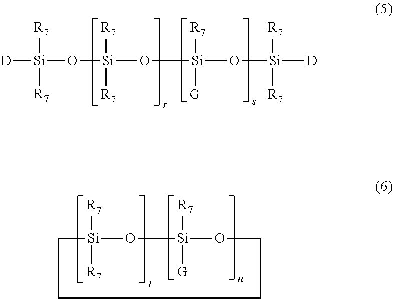

in which
the radicals R⁷, which may be identical or different, are each a C₁-C₁₀ alkyl, phenyl, 3,3,3-trifluoropropyl or trimethylsilyloxy radical, at least 80% by number of the radicals R⁷ being methyl radicals;
the radicals D, which may be identical or different, are each a radical R⁷ or a radical G;
r is an integer ranging from 0 to 50, inclusive, and s is an integer ranging from 0 to 20, inclusive, with the proviso that if s=0, at least one of the two radicals D is a radical G;
u is an integer ranging from 1 to 6, inclusive, and t is an integer ranging from 0 to 10, inclusive, with the proviso that t+u is equal to or greater than 3; and the radical G has the structural formula (2) above.

As can be seen from formula (2) given above, bonding of the divalent radical —(X)ₘ—(CH₂)ₚ—CH(Z)—CH₂— to the benzotriazole group, which thus ensures attachment of said benzotriazole group directly to a silicon atom, may be at any and all of the available positions of the two aromatic rings of the benzotriazole:

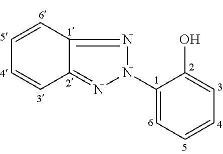 [SMILES: Oc1ccccc1-n1nc2ccccc2n1]

Preferably, this bonding is at position 3, 4, 5 (aromatic ring bearing the hydroxyl function) or 4′ (benzene ring adjacent to the triazole ring), and even more preferably is at position 3, 4 or 5. In a more preferred embodiment of the invention, the bonding is at position 3.

Similarly, attachment of the substituent unit or units Y may be at any and all of the other positions available in the benzotriazole. However, preferably, this bonding is at position 3, 4, 4′, 5 and/or 6. In a more preferred embodiment of the present invention, bonding of the radical Y is at position 5.

In formulae (5) and (6) above, the alkyl radicals may be linear or branched and are selected, in particular, from among methyl, ethyl, n-propyl, isopropyl, n-butyl, isobutyl, tert-butyl, n-amyl, isoamyl, neopentyl, n-hexyl, n-heptyl, n-octyl, 2-ethylhexyl and tert-octyl radicals. The preferred alkyl radicals R⁷ according to the present invention are methyl, ethyl, propyl, n-butyl, n-octyl and 2-ethylhexyl radicals. Even more preferably, the radicals R⁷ are all methyl radicals.

Among the compounds of formula (5) or (6) above, preferred are those corresponding to formula (5), namely, diorganosiloxanes containing a short linear chain.

Among the compounds of formula (5) above, preferred are those for which the radicals D are both radicals R⁷.

Among the linear diorganosiloxanes according to formula (5), more particularly preferred are random derivatives or well-defined block derivatives having at least one, and even more preferably all, of the following characteristics:
D is a radical R⁷;
R⁷ is an alkyl radical and even more preferably is methyl;
r ranges from 0 to 15, inclusive;
s ranges from 1 to 10, inclusive;
n is other than zero and preferably is equal to 1, and Y is then a methyl, tert-butyl or C₁-C₄ alkoxy radical;
Z is hydrogen or methyl;
m=0 or [m=1 and X=0]; and
p is equal to 1.

One family of benzotriazole silicones which is particularly suitable according to the present invention is that having the structural formula (7) below:

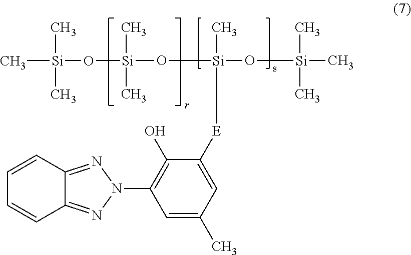

in which
0≤r≤10; 1S≤s≤10; and
E is the divalent radical:

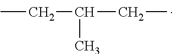

In a particularly preferred embodiment of the present invention, the benzotriazole silicone is the compound Drometrizole Trisiloxane (CTFA name) corresponding to the following structural formula:

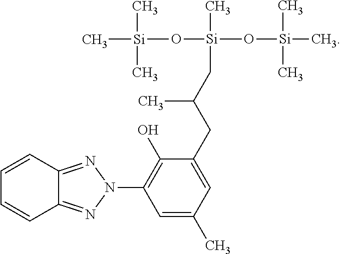 [SMILES: Cc1cc(CC(C)C[Si](C)(O[Si](C)(C)C)O[Si](C)(C)C)c(O)c(-n2nc3ccccc3n2)c1]

Processes which are suitable for the preparation of the compounds of formulae (1), (5), (6) and (7) above are described, in particular, in U.S. Pat. Nos. 3,220,972, 3,697,473, 4,340,709, 4,316,033 and 4,328,346, and also in EP-A-0,392,883 and EP-A-0,742,003.

The amount of the (b) organic UV filter(s) in the composition according to the present invention may be 1% by weight or more, preferably 2% by weight or more, and more 15 preferably 3% by weight or more, relative to the total weight of the composition.

The amount of the (b) organic UV filter(s) in the composition according to the present invention may be 20% by weight or less, preferably 15% by weight or less, and more preferably 10% by weight or less, relative to the total weight of the composition.

The amount of the (b) organic UV filter(s) in the composition according to the present invention may be 1% to 20% by weight, preferably from 2% to 15% by weight, and more preferably from 3% to 10% by weight, relative to the total weight of the composition.

### [Fatty Material]

The composition according to the present invention may comprise (c) at least one fatty material. If two or more fatty materials are used, they may be the same or different.

The term “fatty material” means an organic compound that is insoluble in water at ordinary temperature (25° C.) and at atmospheric pressure (760 mmHg) (solubility of less than 5%, preferably 1% and even more preferentially 0.1%). The fatty material may contain, in its structure, a sequence of at least two siloxane groups or at least one hydrocarbon-based chain containing at least 6 carbon atoms. In addition, the fatty substances may be soluble in organic solvents under the same temperature and pressure conditions, for instance chloroform, ethanol, benzene or decamethylcyclopentasiloxane.

In the scope of the present invention, it has to be noted that the fatty material does not comprise any C₂-C₃ oxyalkylene units or any glycerolated units.

The (c) fatty material may be in the form of a liquid or a solid. Here, “liquid” and “solid” mean that the fatty material is in the form of a liquid or a paste (non-solid) or solid, respectively, at ambient temperature (25° C.) under atmospheric pressure (760 mmHg or 105 Pa). It is preferable that the (c) fatty material comprise at least one fatty material in the form of a liquid or a paste, preferably in the form of a liquid, at ambient temperature and under atmospheric pressure. The (c) fatty material in the form of a liquid at ambient temperature under atmospheric pressure can be referred to as “oil”. It is preferable that the (c) fatty material be selected from oils.

If the (c) fatty material is selected from oils, the composition according to the present invention can have an oil phase or oil phases.

If the (c) fatty material is selected from oils, the (c) fatty material may be selected from polar oils, non-polar oils, and mixtures thereof. It is preferable that the (c) fatty material be selected from polar oils.

The (c) fatty material may be selected from the group consisting of oils of animal or plant origin, mineral oils, synthetic glycerides, esters of fatty alcohols and/or fatty acids other than animal or plant oils and synthetic glycerides, fatty alcohols, fatty acids, silicone oils and hydrocarbons. These fatty materials may be volatile or non-volatile. Preferably, the (c) fatty material is selected from the group consisting of oils of animal or plant origin, synthetic glycerides, fatty esters other than animal or plant oils and synthetic glycerides, fatty alcohols, fatty acids, silicone oils, and hydrocarbons. More preferably, the (c) fatty material is selected from silicones and hydrocarbons, preferably aliphatic hydrocarbons, and more preferably mineral oils, and mixtures thereof.

As examples of aliphatic hydrocarbons, mention may be made of, for example, linear or branched hydrocarbons such as mineral oil (e.g., liquid paraffin), paraffin, vaseline or petrolatum, naphthalenes, and the like; hydrogenated polyisobutene, isoeicosan, polydecenes, hydrogenated polyisobutenes such as Parleam, and decene/butene copolymer; and mixtures thereof.

As examples of other aliphatic hydrocarbons, mention may also be made of linear or branched, or possibly cyclic C₆-C₁₆ lower alkanes. Examples that may be mentioned include hexane, undecane, dodecane, tridecane and isoparaffins such as isohexadecane and isodecane.

As examples of synthetic glycerides, mention may be made of, for instance, caprylic/capric acid triglycerides, for instance those sold by the company, Stearineries Dubois or those sold under the names Miglyol® 810, 812 and 818 by the company, Dynamit Nobel.

As examples of silicone oils, mention may be made of, for example, linear organopolysiloxanes such as dimethylpolysiloxanes or dimethicones, methylphenylpolysiloxanes, methylhydrogenpolysiloxanes, and the like; cyclic organopolysiloxanes such as octamethylcyclotetrasiloxane, decamethylcyclopentasiloxane, dodecamethylcyclohexasiloxane, and the like; and mixtures thereof.

As examples of plant oils, mention may be made of, for example, linseed oil, camellia oil, macadamia nut oil, sunflower oil, apricot oil, soybean oil, arara oil, hazelnut oil, corn oil, mink oil, olive oil, avocado oil, sasanqua oil, castor oil, safflower oil, jojoba oil, sunflower oil, almond oil, grapeseed oil, sesame oil, soybean oil, peanut oil, and mixtures thereof.

As examples of animal oils, mention may be made of, for example, squalene, perhydrosqualene and squalane.

As examples of the esters of a fatty acid and/or of a fatty alcohol, which are advantageously different from the animal or plant oils as well as the synthetic glycerides mentioned above, mention may be made especially of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra-or pentahydroxy alcohols may also be used.

The following may especially be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferable to use ethyl, isopropyl, myristyl, cetyl or stearyl palmitate, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The composition may also comprise, as the fatty ester, sugar esters and diesters of C₆-C₃₀ and preferably C₁₂-C₂₂ fatty acids. The term “sugar” means oxygen-bearing hydrocarbon-based compounds containing several alcohol functions, with or without aldehyde or ketone functions, and which contain at least 4 carbon atoms. These sugars may be monosaccharides, oligosaccharides or polysaccharides.

Examples of suitable sugars that may be mentioned include sucrose (or saccharose), glucose, galactose, ribose, fructose, maltose, mannose, arabinose, xylose and lactose, and derivatives thereof, especially alkyl derivatives, such as methyl derivatives, for instance methylglucose.

The sugar esters of fatty acids may be selected especially from the group comprising the esters or mixtures of esters of sugars described previously, and of linear or branched, saturated or unsaturated C₆-C₃₀ and preferably C₁₂-C₂₂ fatty acids. If they are unsaturated, these compounds may comprise one to three conjugated or non-conjugated carbon-carbon double bonds.

The esters according to this variant may also be selected from mono-, di-, tri-, tetraesters and polyesters, and mixtures thereof.

These esters may be selected, for example, from oleates, laurates, palmitates, myristates, behenates, cocoates, stearates, linoleates, linolenates, caprates and arachidonates, or mixtures thereof such as, especially, oleo-palmitate, oleo-stearate and palmito-stearate mixed esters.

It is more particularly preferable to use monoesters and diesters and especially sucrose, glucose or methylglucose mono- or dioleates, stearates, behenates, oleopalmitates, linoleates, linolenates and oleostearates.

An example that may be mentioned is the product sold under the name Glucate® DO, which is a methylglucose dioleate, by the company Amerchol.

Examples of esters or mixtures of esters of sugar and of fatty acid that may also be mentioned include:
the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referred to as B370 and corresponding to sucrose behenate formed from 20% monoester and 80% di- triester-polyester;
the sucrose mono-dipalmito-stearate sold by the company Goldschmidt under the name Tegosoft® PSE.

The fatty material may be at least one fatty acid, and two or more fatty acids may be used.

The fatty acids should be in acidic form (i.e., unsalified, to avoid soaps) and may be saturated or unsaturated and contain from 6 to 30 carbon atoms and in particular from 9 to 30 carbon atoms, which are optionally substituted, in particular with one or more hydroxyl groups (in particular 1 to 4). If they are unsaturated, these compounds may comprise one to three conjugated or non-conjugated carbon-carbon double bonds. They are more particularly selected from myristic acid, palmitic acid, stearic acid, behenic acid, oleic acid, linoleic acid, linolenic acid and isostearic acid. Preferably the fatty material is not a fatty acid.

The fatty material may be at least one fatty alcohol, and two or more fatty alcohols may be used.

The term “fatty alcohol” here means any saturated or unsaturated, linear or branched C₈-C₃₀ fatty alcohol, which is optionally substituted, in particular with one or more hydroxyl groups (in particular 1 to 4). If they are unsaturated, these compounds may comprise one to three conjugated or non-conjugated carbon-carbon double bonds.

Among the C₅-C₃₀ fatty alcohols, C₁₂-C₂₂ fatty alcohols, for example, are used. Mention may be made among these of lauryl alcohol, cetyl alcohol, stearyl alcohol, isostearyl alcohol, oleyl alcohol, behenyl alcohol, linoleyl alcohol, undecylenyl alcohol, palmitoleyl alcohol, linolenyl alcohol, myristyl alcohol, arachidonyl alcohol and erucyl alcohol, and mixtures thereof. In one embodiment, cetyl alcohol, stearyl alcohol or a mixture thereof (e.g., cetearyl alcohol), as well as myristyl alcohol, can be used as a solid fatty material. In another embodiment, isostearyl alcohol can be used as a liquid fatty material.

The fatty material may be a wax. Here, “wax” means that the fatty material is substantially in the form of a solid at room temperature (25° C.) under atmospheric pressure (760 mmHg), and has a melting point generally of 35° C. or more. As the waxy fatty material, waxes generally used in cosmetics can be used alone or in combinations thereof.

For example, the wax may be selected from carnauba wax, microcrystalline waxes, ozokerites, hydrogenated jojoba oil, polyethylene waxes such as the wax sold under the name “Performalene 400 Polyethylene” by the company New Phase Technologies, silicone waxes, for instance poly(C₂₄-C₂₈)alkylmethyldimethylsiloxane, such as the product sold under the name “Abil Wax 9810” by the company Goldschmidt, palm butter, the C₂₀-C₄₀ alkyl stearate sold under the name “Kester Wax K82H” by the company Kester Keunen, stearyl benzoate, shellac wax, and mixtures thereof. For example, a wax selected from carnauba wax, candelilla wax, ozokerites, hydrogenated jojoba oil and polyethylene waxes can be used. In at least one embodiment, the wax is preferably selected from candelilla wax and ozokerite, and mixtures thereof.

It is preferable that the (c) fatty material be selected from ester oils, fatty alcohols, and a mixture thereof. It is more preferable that the ester oils be chosen from monoesters, diesters and a mixture thereof, which are in the form of an oil.

The amount of the (c) fatty material(s) in the composition according to the present invention may be 1% by weight or more, preferably 3% by weight or more, and more preferably 5% by weight or more, relative to the total weight of the composition.

The amount of the (c) fatty material(s) in the composition according to the present invention may be 20% by weight or less, preferably 15% by weight or less, and more preferably 10% by weight or less, relative to the total weight of the composition.

The amount of the (c) fatty material(s) in the composition according to the present invention may be from 1% to 20% by weight, preferably from 3% to 15% by weight, and more preferably from 5% to 10% by weight, relative to the total weight of the composition.

### [Phosphate Compound]

The composition according to the present invention comprises (d) at least one phosphate compound. Two or more (d) phosphate compounds may be used in combination. Thus, a single type of a phosphate compound or a combination of different types of phosphate compounds may be used.

The term “phosphate compound” here means a compound which has at least one phosphate group.

The “phosphate compound” may have at least one phosphoric acid group, in particular one phosphoric acid group.

The phosphate compound may be selected from organic phosphates, inorganic phosphates and mixtures thereof.

It may be preferable that the inorganic phosphate be selected from inorganic salts of phosphoric acid, such as metal or ammonium salts of phosphoric acid.

It may be more preferable that the inorganic phosphate be in the form of a metal salt. The metal salt may be alkaline metal salts. The alkaline metal salt may be selected from alkaline metal salts of phosphoric acid, such as mono-, di- or tri-sodium phosphate, and mono-, di-, or tri-potassium phosphate. Dipotassium phosphate may be even more preferable.

It may also be preferable that the organic phosphate be selected from esters of phosphoric acid, such as mono-, di- or tri-organophosphates.

According to one embodiment, the (d) phosphate compound may be selected from the group consisting of
(1) the compounds of formula (XIV):

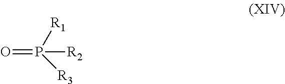

with R₁, R₂ and R₃, which may be identical or different, chosen from:
a group OM with M representing an alkali metal such as Na, Li or K, preferably Na or K,
a group OR₄, in which R₄ represents a linear, branched, cyclic or aromatic C₅-C₄₀ alkyl group, preferably a C₁₂-C₂₀ alkyl group, and more preferably a C₁₆ or C₁₈ alkyl group,
an OH group, and
an oxyethylene group (OCH₂CH₂)ₙ(OCH₂CHCH₃)ₘOR with R representing a hydrogen atom or a linear or branched C₁-C₂₀ alkyl group, for example a C₅-C₁₈ and a C₁₂-C₁₅ alkyl group, and n and m being integers with n ranging from 1 to 50, or being equal to 10, and m ranging from 0 to 50, or being equal to 0,
(2) glycerophospholipids, and
(3) mixtures thereof

It may be preferable that, in the above formula (XIV), at least one of R₁, R₂ and R₃ is a group —OM and at least one of R₁, R₂ and R₃ is a group —OR₄ or —(OCH₂CH₂)ₙ(OCH₂CH(CH₃))ₘOR wherein n and m denote as defined above.

It may also be preferable that, in the above formula (XIV), at least one of R₁, R₂ and R₃ is a group —OM, at least one of R₁, R₂ and R₃ is a group —OH, and at least one of R₁, R₂ and R₃ is a group —OR₄ or —(OCH₂CH₂)ₙ(OCH₂CH(CH₃))ₘOR wherein n and m denote as defined above.

It may also be preferable that, in the above formula (XIV), one of R₁, R₂ and R₃ is a group —OM, one of R₁, R₂ and R₃ is a group —OH, and one of R₁, R₂ and R₃ is a group —OR₄ or -(OCH₂CH₂)ₙ(OCH₂CH(CH₃))ₘOR wherein n and m denote as defined above.

It may also be preferable that, in the above formula (XIV), at least one, or two, or even all of the radicals R₁, R₂ and R₃ contain(s) a group —OR₄, in which R₄ represents a linear or branched C₁₀-C₃₀, in particular C₁₅-C₂₀ or even C₁₆ or C₁₈ alkyl group. In this case, R₁, R₂ and R₃ may be identical or different.

As non-limiting illustrations of these compounds of formula (XIV), mention may be made, for example, of trioleyl phosphate such as Nikkol TDP sold by the company Nikko Chemicals, the mixture of triesters of phosphoric acid and of an ether of ethylene glycol and of C₁₂-C₁₅ fatty alcohols (about 10 EO) (INCI name: Tri-C₁₂₋₁₅ Pareth-10 phosphate) such as Nikkol TDP-10 sold by the company Nikko Chemicals, potassium cetyl phosphate such as Amphisol K sold by the company DSM Nutritional Products or Arlatone MAP 160 K from Uniqema, and ceteth-10 phosphate such as Protaphos CET-10 by the company Protameen Chemicals.

It may be preferable that the (d) phosphate compound has the function of a surfactant. Thus, the (d) phosphate compound may be a phosphate surfactant.

The (d) phosphate compound may be selected from the group consisting of monoester phosphate of alkoxylated fatty alcohol containing from 12 to 20 carbon atoms with from 1 to 50 moles of alkylene oxide selected from ethylene oxide and propylene oxide, and dialkyl phosphates of non-alkoxylated alcohol containing 12 to 22 carbon atoms, and mixtures thereof. The alkyl moiety of the fatty alcohol or the non-alkoxylated alcohol may be a linear or branched, or saturated or unsaturated alkyl group.

The (d) phosphate compound may be selected from the group consisting of a combination of ceteth-10 phosphate and dicetyl phosphate, a combination of ceteth-20 phosphate and dicetyl phosphate, and a combination of oleth-5 phosphate and dioleyl phosphate.

As a product including a combination of ceteth-10 phosphate and dicetyl phosphate, mention may be made of CRODAFOS CES, marketed by Croda Inc., U.S.A. As a product including a combination of ceteth-20 phosphate and dicetyl phosphate, mention may be made of CRODAFOS CS-20 ACID, marketed by Croda Inc., U.S.A. As a product including a combination of oleth-5 phosphate and dioleyl phosphate, mention may be made of CRODAFOS HCE, marketed by Croda Inc., U.S.A.

For the purposes of the present invention, the term “glycerophospholipid” is intended to denote an ester obtained by reacting glycerol with at least one saturated or unsaturated fatty acid and phosphoric acid, the said phosphoric acid being substituted with a compound chosen from alcohols bearing an amine function, especially a β-amino alcohol. The β-amino alcohol may be chosen, for example, from choline, ethanolamine and/or serine.

The glycerophospholipid may be defined according to the general formula (XV) below:

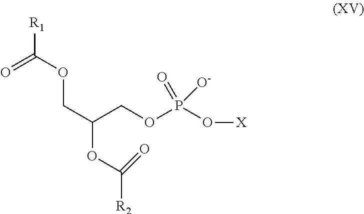

in which:
R₁ and R₂ represent, independently of each other, a saturated or unsaturated, optionally branched fatty acid containing from 4 to 24 carbon atoms, and possibly substituted with one or more hydroxyl and/or amine functions, and
X represents a substituent of general formula R₃R₄R₅N⁺—CH(R₆)—CH₂— in which R₃, R₄, R₅ and
R₆ represent, independently of each other, a hydrogen atom, alkyl groups containing from 1 to 6 carbon atoms, and/or a carboxyl function. X may especially be chosen from choline, serine and ethanolamine.

According to one embodiment, R₁ and R₂, independently of each other, are advantageously chosen from butyric acid, caproic acid, caprylic acid, capric acid, caproleic acid, lauric acid, lauroleic acid, myristic acid, myristoleic acid, palmitic acid, palmitoleic acid, stearic acid, oleic acid, linoleic acid, linolenic acid, arachidic acid, isostearic acid, dihydroxystearic acid and ricinoleic acid.

The glycerophospholipid may also be a mixture of compounds of general formula (XV).

The glycerophospholipid that is suitable for use in the invention may, for example, comprise phosphatidylcholine, phosphatidylethanolamine and/or phosphatidylserine.

According to one embodiment, the glycerophospholipid may comprise an ester of glycerol, of unsaturated fatty acid, of phosphoric acid and of choline, also known as phosphatidylcholine (PC).

The glycerophospholipid that is suitable for use in the invention may be derived from lecithin. The lecithin may comprise, predominantly, phosphatidylcholine as glycerophospholipid.

The phosphatidylcholine (PC) that is suitable for use in the compositions in accordance with the invention may be of “natural” or “synthetic” origin.

“Natural” PC may be obtained by extraction from animal or plant sources, for instance soybean, sunflower or egg. The non-hydrogenated phosphatidylcholine obtained naturally, for instance from soybean, generally contains as glycerol-esterifying fatty acid palmitic acid, stearic acid, palmitoleic acid, oleic acid, linoleic acid, linolenic acid and, optionally, C₂₀-C₂₂ fatty acids.

For the purposes of the present invention, the term “synthetic phosphatidylcholine” is intended to denote phosphatidylcholine comprising at least one fatty acid different from those that may be present in natural PCs.

The term “synthetic PC” is also intended to denote natural PC subjected to modifications, such as partial hydrogenation, i.e. only a fraction of the double bonds present in the unsaturated fatty acids is maintained.

Among the sources of more or less purified phosphatidylcholine that are suitable for use in the cosmetic compositions in accordance with the present invention, mention may be made of Emulmetik 930 sold by the company Lucas Meyer.

The glycerophospholipid that is suitable for use in the present invention may be introduced into the composition in the form of a lecithin. This lecithin is generally obtained by lipid extraction using apolar solvents, from plant or animal fats. This lipid fraction usually comprises, predominantly, glycerophospholipids including phosphatidylcholine or phosphatidylethanolamine.

The lecithins that are suitable for use in the present invention may be lecithins derived from soybean, from sunflower or from egg, and/or mixtures thereof.

Lecithins are usually provided in dissolved form in fatty acids, triglycerides or other solvents, or in the form of powders or cakes.

They are usually mixtures of lecithins, in which the glycerophospholipid content in the products as sold generally ranges from about at least 15% to about at least 95%.

In an exemplary embodiment, the lecithin used as starting material for the preparation of the 25 composition according to the invention comprises at least 45% by weight, for example at least 65% by weight, for example at least 75% by weight, for example at least 85% by weight, or for example at least 95% by weight of glycerophospholipid relative to the total weight of the lecithin.

Among the lecithins that may be suitable for use in the cosmetic compositions in accordance with the present invention, mention may be made of the lecithins sold under the references Nattermann Phospholipid®, Phospholipon 80® and Phosale 75® by the company American Lecithin Company, and Epikuron 145V, Topcithin 300, Emulmetik 930 and Ovothin 200 sold by the company Lucas Meyer.

The glycerophospholipid may be a non-hydrogenated glycerophospholipid, i.e. an ester obtained by reacting glycerol with at least one unsaturated fatty acid and phosphoric acid, said phosphoric acid being substituted with a compound chosen from alcohols bearing an amine function, especially a β-amino alcohol.

The terms “unsaturated” and “unsaturation” are intended to denote the presence of at least one, or even several, double or triple bonds between two carbon atoms.

The glycerophospholipid may be phosphatidylcholine or lecithin.

The (d) phosphate compound may be chosen from trioleyl phosphate, the mixture of triesters of phosphoric acid and of an ether of ethylene glycol and of C₁₂-C₁₅ fatty alcohols (about 10 EO), potassium cetyl phosphate, ceteth-10 phosphate, dipotassium phosphate, cetyl phosphate and lecithin, and mixtures thereof.

The (d) phosphate compound may be a nonionic surfactant such as trioleyl phosphate or the mixture of triesters of phosphoric acid and of an ether of ethylene glycol and of C₁₂-C₁₅ fatty alcohols (about 10 EO).

The amount of the (d) phosphate compound(s) in the composition according to the present invention may be 0.01% by weight or more, preferably 0.05% by weight or more, and more preferably 0.1% by weight or more, relative to the total weight of the composition.

The amount of the (d) phosphate compound(s) in the composition according to the present invention may be 15% by weight or less, preferably 10% by weight or less, and more preferably 5% by weight or less, relative to the total weight of the composition.

The amount of the (d) phosphate compound(s) in the composition according to the present invention may be 0.01% to 15% by weight, preferably from 0.05% to 10% by weight, and more preferably from 0.1% to 5% by weight, relative to the total weight of the composition.

### [Water]

The composition according to the present invention may include (e) water.

The amount of (e) water in the composition according to the present invention may be 40% by weight or more, preferably 45% by weight or more, and more preferably 50% by weight or more, relative to the total weight of the composition.

On the other hand, the amount of (e) water in the composition according to the present invention may be 90% by weight or less, preferably 85% by weight or less, and more preferably 80% by weight or less, relative to the total weight of the composition.

The amount of (e) water in the composition according to the present invention may range from 40% to 90% by weight, preferably from 45% to 85% by weight, more preferably from 50% to 80% by weight, relative to the total weight of the composition.

### [Optional Ingredients]

The composition according to the present invention may include at least one optional ingredient typically employed in cosmetics, specifically, such as dyes, fillers, surfactants or emulsifiers other than the ingredient (d), thickeners, film-forming polymers, natural extracts derived from animals or vegetables, and the like, within a range which does not impair the effects of the present invention.

The composition according to the present invention may include one or several cosmetically acceptable organic solvents, which may be alcohols: in particular monovalent alcohols such as ethyl alcohol, isopropyl alcohol, benzyl alcohol, and phenylethyl alcohol; diols such as ethylene glycol, propylene glycol and caprylyl glycol; other polyols such as glycerol; and ethers such as ethylene glycol monomethyl, monoethyl, and monobutyl ethers, propylene glycol monomethyl, monoethyl, and monobutyl ether, and butylene glycol monomethyl, monoethyl, and monobutyl ethers.

The amount of the organic solvent(s) in the composition according to the present invention 50 may be 0.01% by weight or more, preferably 0.1% by weight or more, and more preferably 1% by weight or more, relative to the total weight of the composition.

On the other hand, the amount of the organic solvent(s) in the composition according to the present invention may be 30% by weight or less, preferably 25% by weight or less, and more preferably 20% by weight or less, relative to the total weight of the composition.

Thus, the amount of the organic solvent(s) in the composition according to the present invention may range from 0.01% to 30% by weight, preferably from 0.1% to 25% by weight, and more preferably from 1% to 20% by weight, relative to the total weight of the composition.

### [Preparation]

The composition according to the present invention can be prepared by mixing the essential ingredient(s) as explained above, and optional ingredient(s), if necessary, as explained above.

The method and means to mix the above essential and optional ingredients are not limited.

Any conventional method and means can be used to mix the above essential and optional ingredients to prepare the composition according to the present invention.

### [Form]

The form of the composition according to the present invention is not limited.

It is preferable that the composition according to the present invention be of the O/W type, more preferably in the form of an O/W emulsion, and even more preferably an O/W gel emulsion.

The O/W architecture or structure, which comprises oil phases dispersed in an aqueous phase, has an external aqueous phase, and therefore products based on the O/W architecture or structure are more pleasant to use because of the feeling of immediate freshness that they can provide.

### [Cosmetic Process and Use]

The composition according to the present invention may be intended to be used as a cosmetic composition. Thus, the cosmetic composition according to the present invention may be intended for application onto a keratin substance. Keratin substance here means a material containing keratin as a main constituent element, and examples thereof include the skin, scalp, nails, lips, hair, and the like. Thus, it is preferable that the cosmetic composition according to the present invention be used for a cosmetic process for the keratin substance, in particular skin.

Thus, the cosmetic composition according to the present invention may be a skin cosmetic composition, and preferably a skin care composition or a skin makeup composition.

The composition according to the present invention can preferably be used as a leave-on or rinse-off cosmetic composition for a keratin substance such as skin. The term “leave-on” here means that the composition according to the present invention is not removed from a keratin substance after being applied onto the keratin substance. The term “rinse-off” here means that the composition according to the present invention is removed, by rinsing, from a keratin substance after being applied onto the keratin substance. Water can be used for rinsing. Even after rinsing off, the composition according to the present invention can remain to form a film or a coating on a keratin substance such as skin.

The present invention also relates to:
a cosmetic process for a keratin substrate such as skin, comprising: applying to the keratin substrate the composition according to the present invention.

The cosmetic process here means non-therapeutic cosmetic method for caring for and/or making up the surface of a keratin substrate such as skin.

Since the composition according to the present invention includes the (a) inorganic UV filter and the (b) organic UV filter, the cosmetic process can protect a keratin substrate such as skin from UV rays, thereby limiting the darkening of the skin, improving the colour and uniformity of the complexion, and/or treating aging of the skin.

The present invention also relates to:
a use of the (b) at least one organic UV filter selected from organosilicon compounds having at least one benzotriazole group in composition comprising the (a) at least one inorganic UV filter in an amount of more than 5% by weight relative to the total weight of the composition, in order to improve the smooth texture of the composition.

The use according to the present invention can reduce a squeaky feeling due to the relatively large amount of the (a) inorganic UV filter in the composition.

The use according to the present invention can provide an improved smooth texture during the application of the composition onto a keratin substance such as skin.

The use according to the present invention can also provide an improved smooth texture even after rinsing off the composition from a keratin substance such as skin.

The explanations regarding the ingredients (a) to (d) for the composition according to the present invention can apply to those in the use according to the present invention.

### EXAMPLES

The present invention will be described in a more detailed manner by way of examples.

However, they should not be construed as limiting the scope of the present invention.

### Examples 1-6 and Comparative Examples 1-271

### [Preparation]

Each of the compositions according to Examples 1-6 and Comparative Examples 1-27 was prepared by mixing the ingredients shown in Tables 1-4 at a room temperature (25° C.). The numerical values for the amounts of the ingredients in Tables 1-4 are all based on “% by weight” as raw materials.

TABLE 1

| | Comp. | | | | | Comp. | | | Comp. |
|---|---|---|---|---|---|---|---|---|---|
| | Ex. 1 | Ex. 1 | Ex. 2 | Ex. 3 | Ex. 4 | Ex. 2 | Ex. 5 | Ex. 6 | Ex. 3 |
| TiO₂ | 7 | 7 | 7 | 7 | 7 | — | 7 | 5 | 3 |
| Drometrizole Trisiloxane (Mexoryl XL) | — | 1 | 2 | 4 | 6 | 6 | 6 | 6 | 6 |
| Ethylhexyl Triazone (Uvinul T150) | — | — | — | — | — | — | 1 | 2 | 3 |
| Potassium Hydroxide | 0.2 | 0.2 | 0.2 | 0.2 | 0.2 | 0.2 | 0.2 | 0.2 | 0.2 |
| Isopropyl Myristate | 8 | 8 | 8 | 8 | 8 | 8 | 8 | 8 | 8 |
| Cetyl Alcohol | 0.7 | 0.7 | 0.7 | 0.7 | 0.7 | 0.7 | 0.7 | 0.7 | 0.7 |
| Diisopropyl Sebacate | 8 | 8 | 8 | 8 | 8 | 8 | 8 | 8 | 8 |
| Silica | 1 | 1 | 1 | 1 | 1 | 1 | 1 | 1 | 1 |
| Xanthan Gum | 0.1 | 0.1 | 0.1 | 0.1 | 0.1 | 0.1 | 0.1 | 0.1 | 0.1 |
| Acrylates/C10-30 Alkyl Acrylate Crosspolymer | 0.35 | 0.35 | 0.35 | 0.35 | 0.35 | 0.35 | 0.35 | 0.35 | 0.35 |
| Poly C10-30 Alkyl Acrylate | 0.35 | 0.35 | 0.35 | 0.35 | 0.35 | 0.35 | 0.35 | 0.35 | 0.35 |
| Phenoxyethanol | 0.6 | 0.6 | 0.6 | 0.6 | 0.6 | 0.6 | 0.6 | 0.6 | 0.6 |
| Ethanol | 3 | 3 | 3 | 3 | 3 | 3 | 3 | 3 | 3 |
| Water | qsp 100 | qsp 100 | qsp 100 | qsp 100 | qsp 100 | qsp 100 | qsp 100 | qsp 100 | qsp 100 |
| Glycerin | 3 | 3 | 3 | 3 | 3 | 3 | 3 | 3 | 3 |
| Propylene Glycol | 3 | 3 | 3 | 3 | 3 | 3 | 3 | 3 | 3 |
| Caprylyl Glycol | 0.3 | 0.3 | 0.3 | 0.3 | 0.3 | 0.3 | 0.3 | 0.3 | 0.3 |
| C12-15 Alkyl Benzoate | 4.8 | 4.8 | 4.8 | 4.8 | 4.8 | 4.8 | 4.8 | 4.8 | 4.8 |
| Stearic Acid | 1.5 | 1.5 | 1.5 | 1.5 | 1.5 | 1.5 | 1.5 | 1.5 | 1.5 |
| Glyceryl Stearate (and) PEG-100 Stearate | 1.5 | 1.5 | 1.5 | 1.5 | 1.5 | 1.5 | 1.5 | 1.5 | 1.5 |
| Potassium Cetyl Phosphate | 1 | 1 | 1 | 1 | 1 | 1 | 1 | 1 | 1 |
| SPF in vitro | 12.6 | 15.0 | 17.1 | 19.5 | 24.4 | 4.2 | 32.3 | 20.3 | 16.8 |
| PPD in vitro | 3.4 | 3.6 | 3.9 | 4.4 | 5.1 | 2.0 | 4.8 | 4.0 | 2.8 |
| Smoothness during application | 3 | 4 | 4 | 5 | 5 | 5 | 4 | 4 | 4 |
| Smoothness after rinsing off | 3 | 4 | 4 | 5 | 5 | 5 | 4 | 5 | 5 |
| Stability | Good | Good | Good | Good | Good | Good | Good | Good | Good |

TABLE 2

| | Comp. | Comp. | Comp. | Comp. | Comp. | Comp. | Comp. | Comp. | Comp. |
|---|---|---|---|---|---|---|---|---|---|
| | Ex. 4 | Ex. 5 | Ex. 6 | Ex. 7 | Ex. 8 | Ex. 9 | Ex. 10 | Ex. 11 | Ex. 12 |
| TiO₂ | 7 | 7 | 7 | 7 | 7 | 7 | 7 | 7 | 7 |
| Ethylhexyl Triazone (Uvinul T150) | 2 | 4 | 6 | — | — | — | — | — | — |
| Octocrylene | — | — | — | 2 | 4 | 6 | — | — | — |
| Octyl Salicylate | — | — | — | — | — | — | 2 | 4 | 6 |
| Potassium Hydroxide | 0.2 | 0.2 | 0.2 | 0.2 | 0.2 | 0.2 | 0.2 | 0.2 | 0.2 |
| Isopropyl Myristate | 8 | 8 | 8 | 8 | 8 | 8 | 8 | 8 | 8 |
| Cetyl Alcohol | 0.7 | 0.7 | 0.7 | 0.7 | 0.7 | 0.7 | 0.7 | 0.7 | 0.7 |
| Diisopropyl Sebacate | 8 | 8 | 8 | 8 | 8 | 8 | 8 | 8 | 8 |
| Silica | 1 | 1 | 1 | 1 | 1 | 1 | 1 | 1 | 1 |
| Xanthan Gum | 0.1 | 0.1 | 0.1 | 0.1 | 0.1 | 0.1 | 0.1 | 0.1 | 0.1 |
| Acrylates/C10-30 Alkyl Acrylate Crosspolymer | 0.35 | 0.35 | 0.35 | 0.35 | 0.35 | 0.35 | 0.35 | 0.35 | 0.35 |
| Poly C10-30 Alkyl Acrylate | 0.35 | 0.35 | 0.35 | 0.35 | 0.35 | 0.35 | 0.35 | 0.35 | 0.35 |
| Phenoxyethanol | 0.6 | 0.6 | 0.6 | 0.6 | 0.6 | 0.6 | 0.6 | 0.6 | 0.6 |
| Ethanol | 3 | 3 | 3 | 3 | 3 | 3 | 3 | 3 | 3 |
| Water | qsp 100 | qsp 100 | qsp 100 | qsp 100 | qsp 100 | qsp 100 | qsp 100 | qsp 100 | qsp 100 |
| Glycerin | 3 | 3 | 3 | 3 | 3 | 3 | 3 | 3 | 3 |
| Propylene Glycol | 3 | 3 | 3 | 3 | 3 | 3 | 3 | 3 | 3 |
| Caprylyl Glycol | 0.3 | 0.3 | 0.3 | 0.3 | 0.3 | 0.3 | 0.3 | 0.3 | 0.3 |
| C12-15 Alkyl Benzoate | 4.8 | 4.8 | 4.8 | 4.8 | 4.8 | 4.8 | 4.8 | 4.8 | 4.8 |
| Stearic Acid | 1.5 | 1.5 | 1.5 | 1.5 | 1.5 | 1.5 | 1.5 | 1.5 | 1.5 |
| Glyceryl Stearate (and) PEG-100 Stearate | 1.5 | 1.5 | 1.5 | 1.5 | 1.5 | 1.5 | 1.5 | 1.5 | 1.5 |
| Potassium Cetyl Phosphate | 1 | 1 | 1 | 1 | 1 | 1 | 1 | 1 | 1 |
| SPF in vitro | 30.1 | 36.1 | 17.0 | 16.1 | 20.4 | 25.4 | 12.7 | 15.1 | 15.9 |
| PPD in vitro | 3.4 | 3.4 | 3.4 | 3.5 | 3.6 | 3.7 | 3.4 | 3.4 | 3.4 |
| Smoothness during application | 3 | 2 | 1 | 3 | 4 | 4 | 3 | 4 | 4 |
| Smoothness after rinsing off | 4 | 4 | 4 | 3 | 3 | 3 | 3 | 3 | 3 |
| Stability | Good | Good | Poor | Good | Good | Good | Good | Good | Good |

TABLE 3

| | Comp. | Comp. | Comp. | Comp. | Comp. | Comp. | Comp. | Comp. | Comp. |
|---|---|---|---|---|---|---|---|---|---|
| | Ex. 13 | Ex. 14 | Ex. 15 | Ex. 16 | Ex. 17 | Ex. 18 | Ex. 19 | Ex. 20 | Ex. 21 |
| TiO₂ | 7 | 7 | 7 | 7 | 7 | 7 | 7 | 7 | 7 |
| Homosalate | 2 | 4 | 6 | — | — | — | — | — | — |
| Ethylhexyl Methoxycinnamate (Parsol MCX) | — | — | — | 2 | 4 | 6 | — | — | — |
| Bis-Ethylhexyloxyphenol Methoxyphenyl | — | — | — | — | — | — | 2 | 4 | 6 |
| Triazine (Tinosorb S) | | | | | | | | | |
| Potassium Hydroxide | 0.2 | 0.2 | 0.2 | 0.2 | 0.2 | 0.2 | 0.2 | 0.2 | 0.2 |
| Isopropyl Myristate | 8 | 8 | 8 | 8 | 8 | 8 | 8 | 8 | 8 |
| Cetyl Alcohol | 0.7 | 0.7 | 0.7 | 0.7 | 0.7 | 0.7 | 0.7 | 0.7 | 0.7 |
| Diisopropyl Sebacate | 8 | 8 | 8 | 8 | 8 | 8 | 8 | 8 | 8 |
| Silica | 1 | 1 | 1 | 1 | 1 | 1 | 1 | 1 | 1 |
| Xanthan Gum | 0.1 | 0.1 | 0.1 | 0.1 | 0.1 | 0.1 | 0.1 | 0.1 | 0.1 |
| Acrylates/C10-30 Alkyl Acrylate Crosspolymer | 0.35 | 0.35 | 0.35 | 0.35 | 0.35 | 0.35 | 0.35 | 0.35 | 0.35 |
| Poly C10-30 Alkyl Acrylate | 0.35 | 0.35 | 0.35 | 0.35 | 0.35 | 0.35 | 0.35 | 0.35 | 0.35 |
| Phenoxyethanol | 0.6 | 0.6 | 0.6 | 0.6 | 0.6 | 0.6 | 0.6 | 0.6 | 0.6 |
| Ethanol | 3 | 3 | 3 | 3 | 3 | 3 | 3 | 3 | 3 |
| Water | qsp 100 | qsp 100 | qsp 100 | qsp 100 | qsp 100 | qsp 100 | qsp 100 | qsp 100 | qsp 100 |
| Glycerin | 3 | 3 | 3 | 3 | 3 | 3 | 3 | 3 | 3 |
| Propylene Glycol | 3 | 3 | 3 | 3 | 3 | 3 | 3 | 3 | 3 |
| Caprylyl Glycol | 0.3 | 0.3 | 0.3 | 0.3 | 0.3 | 0.3 | 0.3 | 0.3 | 0.3 |
| C12-15 Alkyl Benzoate | 4.8 | 4.8 | 4.8 | 4.8 | 4.8 | 4.8 | 4.8 | 4.8 | 4.8 |
| Stearic Acid | 1.5 | 1.5 | 1.5 | 1.5 | 1.5 | 1.5 | 1.5 | 1.5 | 1.5 |
| Glyceryl Stearate (and) PEG-100 Stearate | 1.5 | 1.5 | 1.5 | 1.5 | 1.5 | 1.5 | 1.5 | 1.5 | 1.5 |
| Potassium Cetyl Phosphate | 1 | 1 | 1 | 1 | 1 | 1 | 1 | 1 | 1 |
| SPF in vitro | 12.7 | 14.3 | 15.5 | 19.8 | 28.0 | 35.3 | 20.8 | 25.1 | 26.0 |
| PPD in vitro | 3.4 | 3.4 | 3.4 | 3.4 | 3.5 | 3.5 | 5.2 | 6.1 | 6.2 |
| Smoothness during application | 3 | 4 | 4 | 3 | 4 | 4 | 3 | 2 | 1 |
| Smoothness after rinsing off | 3 | 3 | 3 | 3 | 3 | 3 | 4 | 4 | 4 |
| Stability | Good | Good | Good | Good | Good | Good | Good | Poor | Poor |

TABLE 4

| | Comp. | Comp. | Comp. | Comp. | Comp. | Comp. |
|---|---|---|---|---|---|---|
| | Ex. 22 | Ex. 23 | Ex. 24 | Ex. 25 | Ex. 26 | Ex. 27 |
| TiO₂ | 7 | 7 | 7 | 7 | 7 | 7 |
| Diethylamino Hydroxybenzoyl Hexyl Benzoate (Uvinul A+) | 2 | 4 | 6 | — | — | — |
| Avobenzone (Parsol 1789) | — | — | — | 2 | 4 | 6 |
| Potassium Hydroxide | 0.2 | 0.2 | 0.2 | 0.2 | 0.2 | 0.2 |
| Isopropyl Myristate | 8 | 8 | 8 | 8 | 8 | 8 |
| Cetyl Alcohol | 0.7 | 0.7 | 0.7 | 0.7 | 0.7 | 0.7 |
| Diisopropyl Sebacate | 8 | 8 | 8 | 8 | 8 | 8 |
| Silica | 1 | 1 | 1 | 1 | 1 | 1 |
| Xanthan Gum | 0.1 | 0.1 | 0.1 | 0.1 | 0.1 | 0.1 |
| Acrylates/C10-30 Alkyl Acrylate Crosspolymer | 0.35 | 0.35 | 0.35 | 0.35 | 0.35 | 0.35 |
| Poly C10-30 Alkyl Acrylate | 0.35 | 0.35 | 0.35 | 0.35 | 0.35 | 0.35 |
| Phenoxyethanol | 0.6 | 0.6 | 0.6 | 0.6 | 0.6 | 0.6 |
| Ethanol | 3 | 3 | 3 | 3 | 3 | 3 |
| Water | qsp 100 | qsp 100 | qsp 100 | qsp 100 | qsp 100 | qsp 100 |
| Glycerin | 3 | 3 | 3 | 3 | 3 | 3 |
| Propylene Glycol | 3 | 3 | 3 | 3 | 3 | 3 |
| Caprylyl Glycol | 0.3 | 0.3 | 0.3 | 0.3 | 0.3 | 0.3 |
| C12-15 Alkyl Benzoate | 4.8 | 4.8 | 4.8 | 4.8 | 4.8 | 4.8 |
| Stearic Acid | 1.5 | 1.5 | 1.5 | 1.5 | 1.5 | 1.5 |
| Glyceryl Stearate (and) PEG-100 Stearate | 1.5 | 1.5 | 1.5 | 1.5 | 1.5 | 1.5 |
| Potassium Cetyl Phosphate | 1 | 1 | 1 | 1 | 1 | 1 |
| SPF in vitro | 15.0 | 17.6 | 20.1 | 17.5 | 21.0 | 21.1 |
| PPD in vitro | 5.7 | 9.0 | 13.5 | 7.3 | 13.0 | 13.1 |
| Smoothness during application | 3 | 2 | 1 | 3 | 2 | 1 |
| Smoothness after rinsing off | 4 | 4 | 4 | 4 | 4 | 4 |
| Stability | Good | Good | Good | Good | Poor | Poor |

### [Evaluations]

(SPF in vitro)

Each of the compositions according to Examples 1-6 and Comparative Examples 1-27 was applied onto a PMMA (polymethylmethacrylate) plate at 25° C. in an amount of 1.3 mg/cm². After drying each composition, the UV absorbance spectrum of the composition was measured with a SPF analyzer (UV-2000S) and a SPF value in vitro thereof was calculated by the UVB absorption intensity measured by the SPF analyzer.

The results are shown in Tables 1-4.

(PPD in vitro)

Each of the compositions according to Examples 1-6 and Comparative Examples 1-27 was applied onto a PMMA (polymethylmethacrylate) plate at 25° C. in an amount of 1.3 mg/cm². After drying each composition, the UV absorbance spectrum of the composition was measured with a SPF analyzer (UV-2000S) and a PPD (Persistent Pigment Darkening) value in vitro thereof was calculated by the UVA absorption/UVB absorption ratio based on the UVA absorption and UVB absorption measured by the SPF analyzer.

The results are shown in Tables 1-4.

### (Smoothness During Application)

Each of the compositions according to Examples 1-6 and Comparative Examples 1-27 was applied onto the underarm of 3 experts with fingers in an amount of 2 mg/cm². The panelists evaluated the smoothness during application with a grade from 1 (very unsmooth=squeaky) to 5 (very smooth), and the scores of the grades were averaged.

The results are shown in Tables 1-4.

(Smoothness after Rising Off)

Each of the compositions according to Examples 1-6 and Comparative Examples 1-27 was applied onto the underarm of 3 experts with fingers in an amount of 2 mg/cm². The panelists evaluated the smoothness after rising the composition off with tap water with a grade from 2 (very unsmooth=squeaky) to 5 (very smooth), and the scores of the grades were averaged.

The results are shown in Tables 1-4.

### (Stability)

After storing at 4° C. for 1 month from the preparation of the compositions according to Examples 1-6 and Comparative Examples 1-27, each of the compositions was subjected to a microscopic observation to check the crystallization of the organic UV filter.

Specifically, a small drop of each of the compositions according to Examples 1-6 and Comparative Examples 1-27 was put on a transparent glass plate and covered with a cover glass, and it was observed with a polarizing microscope (Olympus BX51).

If no crystal was found, it was evaluated as Good.

If any crystal was found, it was evaluated as Poor.

The results are shown in Tables 1-4.

### SUMMARY

The compositions according to Examples 1-6 can provide sufficient UV protection regarding both UVA and UVB as well as a smooth feeling to touch both during application and after rinsing off, even though they include a relatively large amount of TiO₂ as the (a) inorganic UV filter.

In addition, the compositions according to Examples 1-6 are stable such that no crystal of drometrizole trisiloxane as the (b) organic UV filter is found therein.

The composition according to Comparative Example 1 does not include drometrizole trisiloxane, and therefore, the UV protection by the composition is not sufficient and the smoothness thereof is not good.

The composition according to Comparative Example 2 does not include TiO₂, and therefore, the UV protection by the composition is not sufficient.

The composition according to Comparative Example 3 includes less than 5% by weight of TiO₂, and therefore, the UV protection by the composition is not sufficient.

The compositions according to Comparative Examples 4-27 do not include drometrizole trisiloxane, and therefore, the smoothness thereof during application or after rinsing off is not good, or the UV protection (in particular against UVA) by the composition is not sufficient.

### Examples 7-111

### [Preparation]

Each of the compositions according to Examples 7-11 was prepared by mixing the ingredients shown in Table 5 at room temperature (25° C.). The numerical values for the amounts of the ingredients in Table 5 are all based on “% by weight” as raw materials.

TABLE 5

| | Ex. 7 | Ex. 8 | Ex. 9 | Ex. 10 | Ex. 11 |
|---|---|---|---|---|---|
| TiO₂ | 7 | 7 | 7.5 | 7.5 | 7.5 |
| Drometrizole Trisiloxane (Mexoryl XL) | 6 | 6 | 6 | 6 | 6 |
| Potassium Hydroxide | 0.2 | 0.2 | 0.2 | 0.2 | 0.2 |
| Niacinamide | 5 | 5 | 5 | 5 | 5 |
| Isopropyl Myristate | 8 | 8 | — | 2 | 8 |
| Cetyl Alcohol | 0.7 | 0.7 | 0.7 | 0.7 | 0.7 |
| Diisopropyl Sebacate | 8 | 8 | 8 | 8 | 8 |
| Silica | 1 | 1 | 1 | 1 | 1 |
| Xanthan Gum | 0.1 | 0.1 | 0.1 | 0.1 | 0.1 |
| Acrylates/C10-30 Alkyl Acrylate Crosspolymer | 0.35 | 0.35 | 0.35 | 0.35 | 0.35 |
| Poly C10-30 Alkyl Acrylate | 0.35 | 0.35 | 0.35 | 0.35 | 0.35 |
| Phenoxyethanol | 0.6 | 0.6 | 0.6 | 0.6 | 0.6 |
| Ethanol | 3 | 3 | 3 | 3 | 3 |
| Water | qsp 100 | qsp 100 | qsp 100 | qsp 100 | qsp 100 |
| Glycerin | 3 | 3 | 3 | 3 | 3 |
| Propylene Glycol | 3 | 3 | 3 | 3 | 3 |
| Caprylyl Glycol | 0.3 | 0.3 | 0.3 | 0.3 | 0.3 |
| C12-15 Alkyl Benzoate | 4.8 | 4.8 | 4.8 | 4.8 | 4.8 |
| Ethylhexyl Triazone (Uvinul T150) | 3 | 3 | 3 | 3 | 3 |
| Diethylamino Hydroxybenzoyl Hexyl Benzoate (Uvinul A+) | — | 1 | — | — | 1 |
| Stearic Acid | 1.5 | 1.5 | 1.5 | 1.5 | 1.5 |
| Glyceryl Stearate (and) PEG-100 Stearate | 1.5 | 1.5 | 1.5 | 1.5 | 1.5 |
| Potassium Cetyl Phosphate | 1 | 1 | 1 | 1 | — |
| Emulsion Stability | Good | Good | Fair | Good | Fair |

### [Evaluation]

### (Emulsion Stability)

After storing at 45° C. for 2 months from the preparation of the compositions according to Examples 7-11, each of the compositions was subjected to a visual observation.

Specifically, 40 g of each of the compositions according to Examples 7-11 was filled into a transparent glass bottle, and was stored at 45° C. for 2 months. The aspect of the transparent glass bottle was visually checked.

If no change was observed, it was evaluated as Good.

If a slight color change or a slight phase separation (e.g, floating oil on top) was found, it was evaluated as Fair.

If a color change or a phase separation was found, it was evaluated as Poor.

The results are shown in Table 5.

### SUMMARY

The compositions according to Examples 7, 8 and 10 are more stable, while the compositions according to Examples 9 and 11 which do not include isopropyl myristate or potassium cetyl phosphate are less stable.

Thus, it is preferable that the composition including TiO₂ and drometrizole trisiloxane also include the (c) fatty material such as isopropyl myristate and/or the (d) phosphate compound such as potassium cetyl phosphate.

1. A composition, comprising:
(a) at least one inorganic UV filter, and
(b) at least one organic UV filter selected from organosilicon compounds having at least one benzotriazole group,
wherein
the amount of the (a) inorganic UV filter is more than 5% by weight relative to the total weigh of the composition.

2. The composition according to claim 1, wherein the (a) inorganic UV filter is selected from titanium dioxide, zinc oxide and a mixture thereof.

3. The composition according to claim 1, wherein the amount of the (a) inorganic UV filter(s) in the composition is 20% by weight or less relative to the total weight of the composition.

4. The composition according to claim 1, wherein the (b) organic UV filter is selected from silanes and polyorganosiloxanes, containing at least one structural unit having the formula (1): 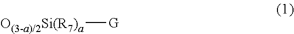
in which
R₇ is an optionally halogenated C₁-C₁₀ alkyl radical, a phenyl radical, or a trimethylsilyloxy radical;
a is an integer ranging from 0 to 3, inclusive; and
G is a monovalent radical directly bonded to a silicon atom and having the structural formula (2): 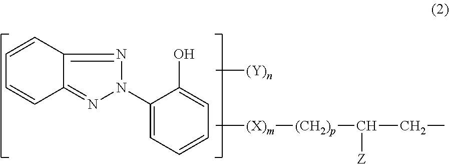
in which
the radicals Y, which may be identical or different, are each a C₁-C₈ alkyl radical, a halogen atom, or a C₁-C₄ alkoxy radical, with the proviso that, in the latter instance, two adjacent radicals Y on the same aromatic ring can together form an alkylidenedioxy radical wherein the alkylidene moiety has 1 or 2 carbon atoms;
X is O or NH;
Z is hydrogen or a C₁-C₄ alkyl radical;
n is an integer ranging from 0 to 3, inclusive;
m is 0 or 1; and
p is an integer ranging from 1 to 10, inclusive.

5. The composition according to claim 1, wherein the (b) organic UV filter is selected from benzotriazole-substituted silicone compounds having either of the structural formulae (5) and (6): 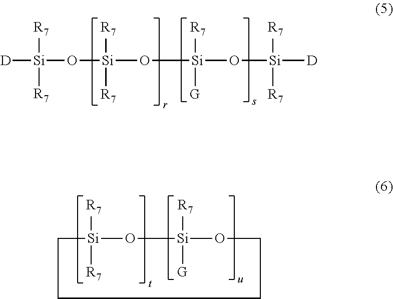
in which
the radicals R⁷, which may be identical or different, are each a C₁-C₁₀ alkyl, phenyl, 3,3,3-trifluoropropyl, or trimethylsilyloxy radical, at least 80% by number of the radicals R⁷ being methyl radicals;
the radicals D, which may be identical or different, are each a radical R⁷ or a radical G;
r is an integer ranging from 0 to 50, inclusive, and s is an integer ranging from 0 to 20, inclusive, with the proviso that if s=0, at least one of the two radicals D is a radical G;
u is an integer ranging from 1 to 6, inclusive, and t is an integer ranging from 0 to 10, inclusive, with the proviso that t+u is equal to or greater than 3; and
the radical G has the structural formula (2): 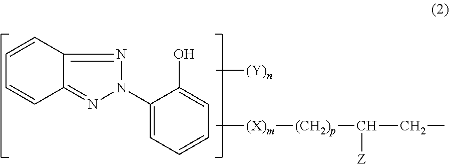
in which
the radicals Y, which may be identical or different, are each a C₁-C₈ alkyl radical, a halogen atom, or a C₁-C₄ alkoxy radical, with the proviso that, in the latter instance, two adjacent radicals Y on the same aromatic ring can together form an alkylidenedioxy radical wherein the alkylidene moiety has 1 or 2 carbon atoms;
X is O or NH;
Z is hydrogen or a C₁-C₄ alkyl radical;
n is an integer ranging from 0 to 3, inclusive;
m is 0 or 1; and
p is an integer ranging from 1 to 10, inclusive.

6. The composition according to claim 1, wherein the (b) organic UV filter is selected from benzotriazole-substituted silicone compounds having the structural formula (7): 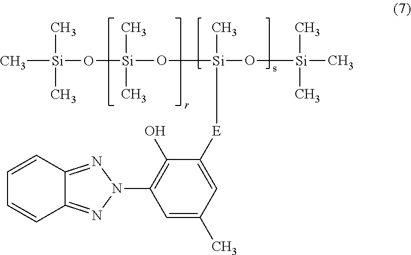
in which 0≤r≤10; 1≤s≤10; and E is the divalent radical:

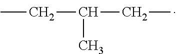

7. The composition according to claim 1, wherein the (b) organic UV filter is drometrizole trisiloxane having the structural formula:

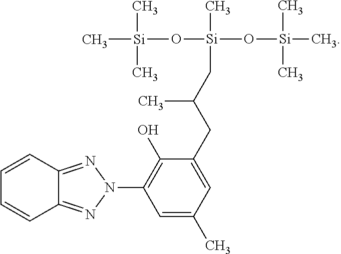 [SMILES: Cc1cc(CC(C)C[Si](C)(O[Si](C)(C)C)O[Si](C)(C)C)c(O)c(-n2nc3ccccc3n2)c1]

8. The composition according to claim 1, wherein the amount of the (b) organic UV filter(s) in the composition is from 1% to 20% by weight relative to the total weight of the composition.

9. The composition according to claim 1, wherein the composition further comprises (c) at least one fatty material.

10. The composition according to claim 9, wherein the amount of the (c) fatty material (s) in the composition is from 1% to 20% by weight relative to the total weight of the composition.

11. The composition according to claim 1, wherein the composition further comprises (d) at least one phosphate compound.

12. The composition according to claim 11, wherein the amount of the (d) phosphate compound(s) in the composition is from 0.01% to 15% by weight relative to the total weight of the composition.

13. The composition according to claim 1, wherein the composition is a cosmetic composition.

14. A cosmetic process for a keratin substrate comprising
applying to the keratin substrate the composition according to claim 1.

15. A method for improving a smooth texture of a composition, comprising:
combining (b) at least one organic UV filter selected from organosilicon compounds having at least one benzotriazole group with a composition comprising (a) at least one inorganic UV filter in an amount of more than 5% by weight relative to the total weight of the composition.
